(19) United States
(12) Patent Application Publication
(10) Pub. No.: US 2006/0002940 A1
(43) Pub. Date: Jan. 5, 2006

(54) Method of immunotherapy

(76) Inventor: Jennifer Stevenson, San Diego, CA (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/507,986

(22) PCT Filed: Mar. 14, 2003
(86) PCT No.: PCT/US03/07983

§ 371 Date: Jun. 29, 2005

Related U.S. Application Data
(60) Provisional application No. 60/364,664, filed on Mar. 15, 2002.

(51) Int. Cl. A61K 39/395; A61K 38/18

(52) U.S. Cl. 424/178.1; 514/7

(57) ABSTRACT

Disclosed are methods and compositions for the treatment of a variety of illnesses by mimicking the outer leaflet of an apotic cell to allow for the induction of macrophage phagocytosis to serve as a means of removing unwanted and diseased cells using phosphatidylserine/cell-surface recognition domain conjugate compositions. Also disclosed are methods for making phosphatidylserine/cell-surface recognition domain conjugate compositions and their formulation for use in a various pharmaceutical applications including the treatment of diverse cancers and other maladies.

### FIELD OF THE INVENTION

The present invention relates generally to immunotherapy and more specifically to selective marking and targeting of cells for destruction by the immune system via phagocytosis using lipid/cell-surface recognition domain conjugate compositions such as phosphatidylserine (PS)-conjugates.

### BACKGROUND INFORMATION

The results of many studies have led to the view that cell surfaces typically display membrane phospholipid asymmetry. While such asymmetry seems to be the rule for normal cells, loss of membrane lipid sidedness, in particular the emergence of phosphatidylserine (PS) at the cell surface, results in the expression of altered surface properties that modulates cell function and influences cell interaction with its environment (Zwaal and Schroit, Blood (1997) 89:1121-1132). Phosphatidylserine exposure has several potential biological consequences, one of which is recognition and removal of the apoptotic cell by phagocytes (Fadok et al., Cell Death Diff (1998) 5:551-562).

Apoptosis is an active, energy-dependent process, which plays an important role in a wide variety of physiological conditions ranging from loss of redundant structures during embryological development to elimination of potentially autoreactive T cells during thymic education. Cell death by apoptosis is a process in which cell deletion within tissues occurs by phagocytosis without evoking an inflammatory response (U.S. Pat. No. 5,776,905).

Exposure of phosphatidylserine on the external leaflet of the plasma membrane appears to be common to many apoptotic cells (Fadok et al., J Immunol (1992a) 148:2207-2216; Fadok et al., J Immunol (1992b) 149:4029-4035; Schlegel et al., Immunol Letts (1993) 36:283-288) and this phospholipid appears to be recognized in a stereoselective manner by macrophages (Fadok et al., 1992a, b; Pradham et al., Mol Bio Cell (1997) 8:767-778), melanoma cells (Fadok, J Cellular Biochem (1995) S19B: 262), smooth muscle vascular cells (Bennett et al., Circ Res (1996) 77:1136-1142) and Sertoli cells (Shriatsuchi et al., J Bio Chem (1997) 272:2354-2358).

It is well recognized that macrophages appear to recognize cells which have lost membrane asymmetry (Fidler et al., Cancer Res (1980) 40:4460-4466; McEvoy et al., Proc Natl Acad Sci USA (1986) 83:3311-3315). Further, investigations have found that human and rodent macrophages preferentially take up negatively charged liposomes, particularly those containing PS (e.g., Fidler et al., (1980); Schroit et al., Cancer Res (1982) 42:161-167; Mehta et al., J. Retuculoendothelial Soc (1992) 32: 155-164). Moreover, studies have demonstrated that human and rodent macrophages can bind to and engulf symmetric red cell ghosts, red cells with PS inserted externally, oxidized red cells or sickled red cells, all of which express PS externally Tanaka et al., J Biol Chem (1983) 258:11335-11343; Hebbel et al., Blood (1984) 64:733-741; Schwartz et al., J Clin Invest (1985) 75:1965-1972; Connor et al., J Biol Chem (1994) 269:2399-2804).

While aminophopholipids (especially PS) have become indispensable tools in the study of membrane structure and function, to include the use of antibodies against PS in diagnosis and treatment modalities (e.g., U.S. Pat. Nos. 6,521,211; 6,524,583; 6,406,693; 6,300,308), little attention has been given to the application of aminophospholipid-specific activation of macrophages as a means for treating disease.

Existing therapies include delivering therapeutic agents that attempt to destroy cells by causing DNA damage and/or interference with metabolism or the cell cycle. Such modalities unfortunately can often be indiscrininant, affecting both normal and diseased cells. Also, many modalities generally require cellular uptake of an agent for therapeutic efficacy, which can lead to the development of defense mechanisms by targeted cells (e.g., the multiple drug resistances (MDR) seen in malignant cells, which can up-regulate transporters that pump chemotherapeutics compounds out of the cell).

Further, many modalities seek to exploit cell surface markers/antigens (e.g., tumor antigens) by situs delivery of modified versions of such markers in order to prompt an immune response from the recipient toward the particular targeted cell. These therapies, however, have not been highly successful (see, e.g., Holmberg et al., Bone Marrow Transpl (2000) 25:1233-1241). Thus, there exists an immediate need for an effective method of exploiting cell surface peculiarities and cell-surface selective immune responses for use in the diagnosis and treatment of various conditions.

### SUMMARY OF THE INVENTION

The present invention overcomes one or more of the drawbacks in the prior art by providing novel compositions and methods for their use in the induction of a macrophage response to lipids such as PS. Disclosed are methods for the preparation and use of lipid compositions which generate a macrophage response to remove targeted cells in an animal. Also disclosed are methods for the use of lipid/cell-surface recognition domain conjugate compositions (specifically those comprising PS) in a variety therapeutic modalities, including the treatment of cancer. Embodiments are disclosed which describe methods for making and using lipid/cell-surface recognition domain conjugate compositions such as phosphatidylserine (PS)-conjugates which allow for extracellular-matrix, PS-specific induction of phagocytes for removal of targeted cells. Also disclosed are methods for making PS-conjugate compositions and their use in a variety of therapeutic applications, including the formulation of pharmaceutical compositions for the prevention and treatment of diseases including viral and bacterial infections, inflammatory disorders, and cancer.

Exemplary methods and compositions according to this invention, are described in greater detail below.

In one embodiment, methods are disclosed for specifically targeting cells for phagocytosis, which include presenting phosphatidylserine on the surface of said cells. Such methods comprise contacting said cells with a bi-functional phosphatidylserine/cell-surface recognition domain conjugate. Contacts are formed by selective interaction of the conjugate with the surface of the target cells by binding to a cognate moiety. Further, such cells may include neoplastic cells, infected cells or select immune cells. In a related aspect, such neoplastic cells can include tumor or metastatic cells. In a further related aspect, infected cells may include bacterially or virally infected cells.

Methods are disclosed for specifically targeting cells for phagocytosis, whereby the conjugate comprises specific stereoisomers of phosphatidylserine that are recognized by macrophages. Further, such macrophages may be activated prior to contacting by the conjugate. For example, enlisting of T_{H}1 cells (e.g., CD4, CD8 T-cells) and/or contacting macrophages with cytokines (e.g., IFNγ, membrane associated TNFα or TNFβ) and bacterial products are envisaged. In a related aspect, IFNγ and bacterial lipopolysaccharide can be used to activate macrophages

Methods are disclosed for specifically targeting cells for phagocytosis, where the cell-surface recognition domain is a polypeptide. While such a recognition domain need not be limited to polypeptides, in a related aspect, the domain may be in the form of an antibody, receptor, ligand or fragments thereof. In a further related aspect, such fragments would comprise, minimally, the necessary functional/structural elements to allow for selective interaction with a cognate moiety, e.g., an Fab fragment/antigen binding pair.

In another related aspect, such antigens may be tumor or viral antigens. For example, such antigens may include, but are not limited to, carcinoembryonic antigen (CEA), E-cadherin mutational hotspot region (ECMHR), CD17-1A antigen, CD52, CD20, HER-2/neu (c-erbB-2), CD33 and chimeric L6 antigen.

Further, methods envisage cell surface domains comprising specific ligands for specific receptors, including epidermal growth factor (EGF), transforming growth factor α (TGFa), urokinase plasminogen activator (uPA), transferrin, folate, adenovirus fiber, malaria es protein, human papilloma virus capsid, lectins and fibroblast growth factor FGF).

Methods of eliminating cells in a subject are also disclosed, including administering a pharmaceutical composition comprising a bi-functional phosphatidylserine/cell-surface recognition domain conjugate to a subject. In a related aspect, such administering induces phagocytosis of specifically targeted cells.

Further, eliminated cells can be associated with particular diseases such as cancers and infections. In a related aspect, cancers can include, but are not limited to, colorectal, gastrointestinal, pancreatic, prostate, breast, lung, hematopoietic, head and neck and stomach. In a further related aspect, such infections can include viral and bacterial infections.

Also disclosed are immunodetection kits comprising, in suitable container means, a cell targeting bi-functional phosphatidylserine/cell-surface recognition domain conjugate and a detection means for detecting the conjugate. Such detection means include labels such as fluorescent or enzymatic labels for example. Optionally, a kit may include an antibody that specifically binds to phosphatidylserine, and an immunodetection reagent

In an important embodiment, the invention provides cell targeting bi-functional phosphatidylserine/cell-surface recognition domain conjugate compositions and means for making and using these compositions. Such a composition is understood to comprise one or more phosphatidylserine compositions that are able to induce a macrophage/phagocytic response in a subject.

Other features and advantages of the invention will be apparent from the following description of the preferred embodiments thereof, and from the claims.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein.

FIG. 1 is an illustration of the bi-functional phosphatidylserine/cell-surface recognition domain conjugate composed of an antibody domain and phosphatidylserine interacting with a target cell (e.g., primary or metastatic tumor cell) and a phagocyte (e.g., macrophage).

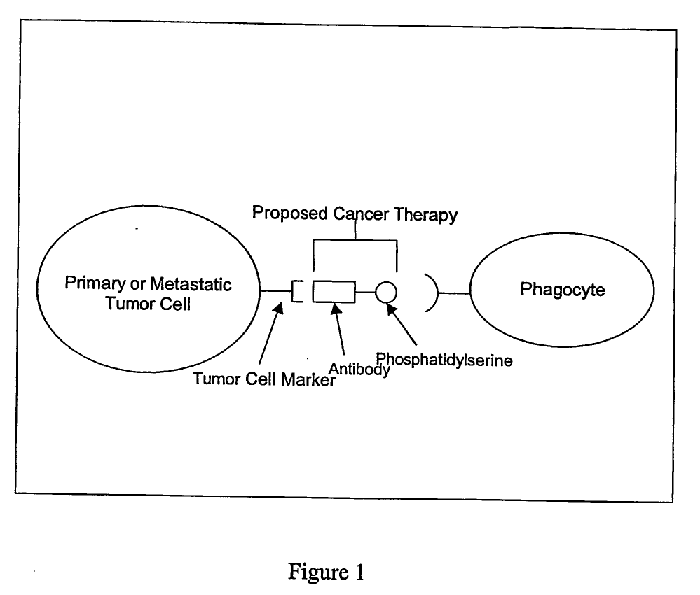

### DETAILED DESCRIPTION OF THE INVENTION

Before the present phospholipids, conjugates, proteins and methods are described, it is understood that this invention is not limited to the particular methodology, protocols, cell lines, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a subject” includes a plurality of such subjects, reference to an “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods, devices, and materials are now described. All publications mentioned herein are incorporated herein by reference for the purpose of describing and disclosing the cell lines, compounds, and methodologies which are reported in the publications which might be used in connection with the invention. Nothing herein is to be construed as an admission that the invention is not entitled to antedate such disclosure by virtue of prior invention.

“Amino acid sequence” as used herein refers to an oligopeptide, peptide, polypeptide, or protein sequence, and fragments or portions thereof, and to naturally occurring or synthetic molecules.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

A “variant”, as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

The term “bifunctional” as used herein refers to relating to, or being a compound with two chemically distinct reactive sites in each molecule.

The term “surface recognition domain” as used herein refers to a separate, physicochemically distinguishable region of a molecule that specifically interacts with the exterior or upper boundary of a cell or object.

The term “cognate” as used herein refers to two molecules that typically interact (e.g., a receptor and its ligand).

The term “biologically active”, as used herein, refers to a protein having structural, regulatory, or biochemical functions of a naturally occurring molecule. Likewise, “immunologically active” refers to the capability of the natural, recombinant, or synthetic molecule, or any oligopeptide thereof, to induce a specific immune response in appropriate animals or cells and to bind with specific antibodies.

The terms “specifically” or “specifically targeting”, as used herein, in reference to the interaction of an antibody/antigen, receptor/cognate ligand, interaction between binding partners (e.g., avidin and biotin, lectins and carbohydrates, protein A and IgG), mean that the interaction is dependent upon the presence of a particular structure (e.g., the antigenic determinant or epitope, stereo-selection); in other words, the one partner of a pair is recognizing and binding to a specific structure rather than to molecules in general (i.e., non-specifically such as with BSA and plastic surfaces). For example, if an antibody is specific for epitope “A”, the presence of a protein containing epitope A (or free, unlabeled A) in a reaction containing labeled “A” and the antibody will reduce the amount of labeled A bound to the antibody.

The present invention provides a means to activate macrophage phagocytosis of phosphatidylserine marked cells using aminophospholipid/cell-surface recognition domain conjugate compositions. Also described is a therapy that targets cells for phagocytosis by presenting phosphatidylserine groups on the surface of the target cells.

The disclosed invention offers that a treatment modality exploiting this mechanism does not cause genetic damage to cells, can be tailored to be selective for specific subsets of cells and avoids the problems associated with conventional drugs that are taken up by the target cells (i.e., drug resistance) or vaccines (i.e., escape by mutational variation).

While T-cell activation of macrophages is not always required for PS recognition-induced ingestion of target cells, in one embodiment enlisting of T_{H}1 cells (e.g., CD4, CD8 T-cells) and/or contacting macrophages with cytokines (e.g., IFγ, membrane associated TNFα or TNFβ and bacterial products are envisaged (Janeway et al., in Immunobiology: The Immune System in Health and Disease. 1999, 4th ed., p. 299, Current Biology Publications, London, UK). In a related aspect, IFNγ and bacterial lipopolysaccharide can be used to activate macrophages. Moreover, digestible particulate stimuli such as β-glucan, zymosin or even apoptotic cells can be used for this purpose (Fadok et al., (1998)).

Mimicking the outer leaflet of an apoptotic cell allows for the exploitation of macrophage phagocytosis to serve as a means of removing unwanted and diseased cells.

As such, the invention disclosure contemplates that the PS compositions described herein may be used for the prevention of or the treatment of essentially any disorder whereby PS can be presented on the surface of the cell.

Such disorders include, but are not limited to, abnormal proliferation of fibrovascular tissue, acquired immune deficiency syndrome, artery occlusion, atopic keratitis, bacterial ulcers, blood borne tumors, carotid obstructive disease, choroidal neovascularization, chronic inflammation, corneal graft rejection, corneal neovascularization, corneal graft neovascularization, Crohn's disease, fungal ulcers, Herpes simplex infections, Herpes zoster infections, hyperviscosity syndromes, Kaposi's sarcoma, leukemia, Lyme's disease, Mycobacteria infections myopia, ocular neovascular disease, osteoarthritis, Pagets disease, protozoan infections, pseudoxanthoma elasticum, pterygium keratitis sicca, retinal neovascularization, retinopathy of prematurity, retrolental fibroplasias, sarcoid, scleritis, sickle cell anemia, solid tumors, syphilis, systemic lupus, toxoplasmosis, trauma, tumors of neuroblastoma, tumors of osteosarcoma, tumors of retinoblastoma, tumors of rhabdomyosarcoma, ulceritive colitis, and non-small cell lung carcinoma.

The present invention further provides methods and uses for the treatment of subjects that have, or are at risk for developing, arthritis.

In one aspect, the treatment may be used to target both infected cells and neoplastic cells. In a related aspect, the treatment may be used to target both primary tumor and metastatic cells.

The conjugate composition comprises a phosphatidylserine-protein conjugate. The protein may be maleimide-activated, or alternatively, may be prepared by introduction of reactive sulfhydryls into the protein. Exemplary proteins contemplated to be useful in the present methods include, but are not limited to, antibodies, receptors, ligands and fragments thereof. As such, a PS composition of the present invention is also understood to comprise one or more PS-containing or other negatively charged formulations that elicit a macrophage specific, phagocytic response in a subject.

The conjugation method may be carried out by a wide variety of methods. In one embodiment, a Schiff base reaction may be used to perform the conjugation (Stretweiser, Jr. et al., in Introduction to Organic Chemistry 1976, Chapter 15, pp. 378-381, MacMillan Publishing Co., Inc., New York, N.Y.). For example, PS can be conjugated with an aldehyde or ketone. The resulting intermediate then reacts with a primary amine (such as those found on proteins), forming a double bond between the carbon on the PS and the nitrogen of the primary amine.

In one embodiment, the method is carried out as described in Diaz et al. (Bioconjugate Chem (1998) 9:250-254), where NH₂-phosphatidylcholine is formed and reacted with N-succinimidyl 3-(2-pyridyldithio) propionate (SPDP) to form SPDP-PC.

In a related aspect, while SPDP is one reagent, N-succinimidyl 4-(p-maleimidophenyl)butyrate (SMPB), N-succinimidyl trans-4-(N-maleimidylmethyl)cyclohexane-1-carboxylate (SMCC), and N-succinimidyl 3-maleimidylbenzoate (SMB) may be used as substitutes (see, e.g., U.S. Pat. No. 6,521,211).

The SPDP-PC intermediate is then converted to SPDP-PS using phospholipase D and subsequently coupled with 2-iminothiolane (2-IT) treated protein.

As stated above, a wide variety of methods are available to one skilled in the art to produce the conjugate. The above examples are by no means exhaustive.

The compounds of the present invention include enantiomers and other similar molecules. The compounds according to this invention contain one or more asymmetric carbon atoms and thus occur as racemates and racemic mixtures, single enantiomers, diastereomeric mixtures and individual diastereomers. The term “stereoisomer” refers to a chemical compound having the same molecular weight, chemical composition, and constitution as another, but with the atoms grouped differently. That is, certain identical chemical moieties are at different orientations in space and, therefore, when pure, have the ability to rotate the plane of polarized light. However, some pure stereoisomers may have an optical rotation that is so slight that it is undetectable with present instrumentation. The compounds described herein may have one or more asymmetrical carbon atoms and therefore include various stereoisomers. All such isomeric forms of these compounds are expressly included in the present invention.

Each stereogenic carbon may be of R or S configuration. Although the specific compounds exemplified in this application may be depicted in a particular configuration, compounds having either the opposite stereochemistry at any given chiral center or mixtures thereof are also envisioned. When chiral centers are found in the derivatives of this invention, it is to be understood that this invention encompasses all possible stereoisomers. The terms “optically pure compound” or “optically pure isomer” refers to a single stereoisomer of a chiral compound regardless of the configuration of the compound.

In one embodiment, the stereochemistry is important in recognition by the phagocyte, as only the L-isomer of PS can inhibit binding of PS to macrophages (Fadok et al., 1998). In a related aspect, depending on the cell-surface recognition domain that is being used, a spacer or linker of variable length may be required to raise the PS away from the cells or to remove the PS from the binding site (avoid steric hindrance) such that the phagocyte is able to recognize the aminophospholipid. The spacer or linker may contain 1 or more carbons, 2 or more carbons, 3 or more carbons or 4 or more carbons

In a further related aspect, the presence of fatty acid side chains is optional as only the glycerol backbone is required for phagocyte recognition. Such a formulation for the PS would be more soluble in water, thus making pharmaceutical formulation and delivery more facile (see, e.g., Diaz et al., 1998).

In another embodiment, the cell-surface recognition domain is not limited to proteins and may comprise other moieties, including carbohydrates and nucleic acids (see, e.g., U.S. Pat. Nos. 6,171,614 and 5,688,941, herein incorporated by reference).

In one embodiment, the cell-surface recognition domain comprises an antibody. Such an antibody may be generated using methods that are well known in the art. Such an antibody may include, but is not limited to, polyclonal, monoclonal, chimeric, single chain, Fab fragments, and fragments produced by an Fab expression library.

In a related aspect, the entire antibody may not be necessary for therapeutic efficacy. For example, using smaller F(ab′)₂ fragment instead of the intact anti-CEA antibodies may result in more facile penetration into tumor masses, thus allowing for higher doses to be given. Further, in another embodiment, recognition domains comprising multiple antibodies or multiple portions of antibodies are also envisaged.

For the production of antibodies, various hosts including goats, rabbits, rats, mice, humans, and others, may be immunized by injection with appropriate antigen or any fragment or oligopeptide thereof which has immunogenic properties. For example, antigens may include, but are not limited to, carcinoembryonic antigen (CEA, Acc. No. S33324), e-cadherin mutational hotspot region (ECMHR, Acc. No.), CD17-1A antigen (Acc. No. P16422), CD52 (Acc. No. NP-001794), CD20 (Acc. No. A30586), HER-2/neu (c-erb-2, Acc. No. AAA58637), CD33 (Acc. No. AAK83654) and chimeric L6 antigen (Acc. No. A53399).

In one embodiment, the antigen is CEA. The human CEA family has been fully characterized. It comprises 29 genes of which 18 are expected; 7 belonging to CEA subgroup and 11 the pregnancy specified glycoprotein subgroup. CEA is an important tumor marker for colorectal and some other carcinomas (Harmmarström, Cancer Biology (1999) 9:67-81).

CEA could be defined as a glycoprotein containing approximately 50% carbohydrate with a molecular weigh of approximately 200 kDa. CEA is expressed in a number of tumors epithelial origin such as colorectal carcinoma, lung adenocarcinoma and mucinous ovarian carcinoma and endometrial adenocarcinoma (Harmmarström, 1999).

Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable. Do we need to specify the timing of delivering the adjuvant (e.g before, simultaneously, or following)?

It is preferred that the peptides, fragments, or oligopeptides used to induce antibodies to antigens have an amino acid sequence consisting of at least five amino acids, and more preferably at least 10 amino acids. It is also preferable that they are identical to a portion of the amino acid sequence of the target protein, and they may contain the entire amino acid sequence of a small, naturally occurring molecule. Short stretches of amino acids may be fused with those of another protein such as keyhole limpet hemocyanin and antibody produced against the chimeric molecule.

Monoclonal antibodies may be prepared using any technique which provides for the production of antibody molecules by continuous cell lines in culture. These include, but are not limited to, the hybridoma technique, the human B-cell hybridoma technique, and the EBV-hybridoma technique (Kohler et al., Nature (1975) 256:495-497; Kozbor et al., J Immunol Methods (1985) 81:31-42; Cote et al., Proc Natl Acad Sci USA (1983) 80:2026-2030; Cole et al., Mol Cell Biol (1984) 62:109-120).

In addition, techniques developed for the production of “chimeric antibodies”, the splicing of mouse antibody genes to human antibody genes to obtain a molecule with appropriate antigen specificity and biological activity can be used (Morrison et al., Proc Natl Acad Sci USA (1984) 81:6851-6855; Neuberger et al., Nature (1984) 312:604-608; Takeda et al., Nature (1985) 314:452-454). Alternatively, techniques described for the production of single chain antibodies may be adapted, using methods known in the art, to produce specific single chain antibodies. Antibodies with related specificity, but of distinct idiotypic composition, may be generated by chain shuffling from random combinatorial immunoglobulin libraries (Burton, Proc Natl Acad Sci USA (1991) 88:11120-3).

Antibodies may also be produced by inducing in vivo production in the lymphocyte population or by screening recombinant immunoglobulin libraries or panels of highly specific binding reagents as disclosed in the literature (Orlandi et al., (1989) Proc Natl Acad Sci USA 86:3833-3837; Winter et al., Nature (1991) 349:293-299).

It is known that antibody fragments may be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using papain produces two monovalent Fab fragments and an Fc fragment directly. These methods are described inter alia in U.S. Pat. No. 4,036,945 and references contained therein, and in Nisonoff et al., Arch Biochem Biophys, (1960) 89:230 (1960); Porter, Biochem J (1959) 73, 119; and Edelman et al., in Methods in Immunology and Immunochemistry, 1967, Vol. 1, p. 422, Academic Press, New York, N.Y.).

In one embodiment, ligand-receptor interaction is envisaged as a means of targeting cell surfaces with the conjugate of the present invention. Cognate ligands for associated receptors include, but are not limited to, epidermal growth factor (EGF, Acc. No. NP-036974[rat]), transforming growth factor α (TGFα, Acc. No. 1103301A[rat]), urokinase plasminogen activator (uPA, Acc. No. NP-037217), transferrin (Acc. No. NP-001054), folate, adenovirus fiber (Acc. No. NP-597753), malaria cs protein (Acc. No. AAA63153), human papilloma virus capsid (Acc. No. AAA46934) and fibroblast growth factor (FGF, Acc. No. CAB61690).

Biologically active receptor polypeptide or fragment thereof is defined herein as a polypeptide that shares an effector function of the mature receptor for the cognate ligands as exemplified, but not limited to, above and that also may (but need not) possess an antigenic function. Thus, the biologically active receptors polypeptides that are the subject of this invention include prepro-receptors; pro-receptors; mature receptors; fragments thereof having a consecutive sequence of at least 5, 10, 15, 20, 25, 30, or 40 amino acid residues from any of the above receptors for the enumerated cognate ligands; amino acid sequence variants of any of these receptors wherein an amino acid residue has been inserted N- or C-terminal to, or within, the prepro-, pro-, pre-, or mature human receptor amino acid sequence or its fragment; amino acid sequence variants of the prepro-, pro-, pre-, or mature receptor amino acid sequence or its fragment as defined above wherein an amino acid residue of any of these sequences or fragment thereof has been substituted by another residue, including predetermined mutations by, e.g., site-directed or PCR mutagenesis, and other mammalian species of the receptor such as human, rabbit, rat, porcine, non-human primate, equine, murine, and ovine version of the receptor and alleles or other naturally occurring variants of the foregoing and human sequences; derivatives of the receptor or its fragments as defined above wherein the receptor or its fragments have been covalently modified, by substitution, chemical, enzymatic, or other appropriate means, with a moiety other than a naturally occurring amino acid; glycosylation variants of the receptor polypeptide for the enumerated cognate receptors enumerated above (insertion of a glycosylation site or deletion of any glycosylation site by deletion, insertion, or substitution of suitable residues); and soluble forms of the receptor such as those that lack a functional transmembrane domain. The preferred homolog receptor polypeptide for the enumerated cognate ligands is the mature receptor polypeptide, more preferably, the human mature receptor.

In another related aspect, conjutgates may be formed between other binding pairs such as PS and lectins, where the latter are specific for carbohydrate structures that are commonly up-regulated in cancer cells (e.g., GM2, GD2, GD3, MBr1 epitope, Fucosyl GM1, 2,8-polysialic acid, Le^{y}, Sialyl Le^{a}, T epitope, T_{N} epitope and STₙ epitope; see, e.g., Livingston et al., Cancer Immunol Immunother (1997) 45(1):1-9).

The present invention also provides articles of manufacture comprising packaging material and a pharmaceutical composition contained within said packaging material, wherein said pharmaceutical composition is capable of treating a pathological condition wherein said packaging material comprises a label which indicates that said pharmaceutical composition can be used for treatment of disorders, and wherein said pharmaceutical composition comprises a compound according to the present invention.

Pharmaceutical compositions employed as a component of invention articles of manufacture can be used in the form of a solid, a solution, an emulsion, a dispersion, a micelle, a liposome, and the like, wherein the resulting composition contains one or more of the compounds described above as an active ingredient, in admixture with an organic or inorganic carrier or excipient suitable for enteral or parenteral applications. Compounds employed for use as a component of invention articles of manufacture may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used.

The present invention also provides pharmaceutical compositions comprising at least one invention compound capable of treating a disorder in an effective amount, and a pharmaceutically acceptable vehicle or diluent. The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation

Invention pharmaceutical compositions may be administered by any suitable means, for example, orally, such as in the form of tablets, capsules, granules or powders; sublingually; buccally, parenterally, such as by subcutaneous, intravenous, intramuscular, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The subjects treated in the above methods, in which cells targeted for destruction is desired, are mammals, including, but not limited to, cows, sheep, goats, horses, dogs, cats, guinea pigs, rats or other bovine, ovine, equine, canine, feline, rodent or murine species, and preferably a human being, male or female.

The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In the treatment of a subject where cells are targeted for destruction, an appropriate dosage level will generally be about 0.01 to 500 mg per kg patient body weight per day which can be administered in single or multiple doses. Preferably, the dosage level will be about 0.1 to about 250 mg/kg per day, more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, once or twice per day, 2-3 times per week or more, once per week, or as needed and determined by the physician

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The invention will now be described in greater detail by reference to the following non-limiting examples.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Production and Characterization of Bi-Functional Phosphatidylserine/Protein Conjugates

The method of synthesis involves the use of synthesized PS that contains a “sulfhydryl-activated” coupling group at the end of the 2-position side chain and covalently links the lipid to a cell surface recognition domain (Diaz et al., 1998).

PS, dioleoylphosphatidic acid (PA), PC, dioleoylphosphatidylglycerol (PG), DOPE is available from Avanti Biochemicals (Pelham, Ala.). 1-acyl-2-(aminocaproyl)phosphatidylcholine (NH₂—PC) is synthesized as previously described (Schroit and Madsen, Biochemistry (1983) 22:3617-3623). N-succinimidyl-3-(2-pyridyldithio) propionate (SPDP) and 2-iminothiolane is available from Pierce (Rockford, Ill.).

Synthesis of 1-Oleoyl-2-N-Succinimidyl-3-(2-Pyridyldithio) Propionyl(Aminocaproyl)-PS(SPDP-PS)

SPDP-PS is made from SPDP-PC by phospholipase D catalyzed base-exchange in the presence of L-serine (Comfurius et al., J Lipid Res (1990) 31:1719-1721). Briefly, SPDP-PC is synthesized first by reacting 20 mmol of NH₂—PC [prepared by deblocking 1-acyl-2-tBOC-aniinocaproyl-PC (Schroit and Madsen, 1983)] with 40 mmol of SPDP in 3 ml of CHCl₃/MEOH/triethylamine (1/2/0.015) overnight. CHCl₃ (1 ml) and water (1.8 ml) is added and the lower organic phase is removed. Analysis of the product, SPDP-PC, is conducted by thin-layer chromatography (ILC) (CHCl₃/MEOH/H₂O; 65/25/4; Rf=about 0.4). Subsequently, the lipid is then dried and resuspended in 1 ml of 50% L-serine in 0.1M acetate buffer, pH 5.6 containing 0.1M CaC₂. 1 ml of ether and 25 units (70 ul) of phospholipase D is added and the suspension is mixed at 45° C. for 3 h and stopped by the addition of EDTA (to 0.2 M). The ether is then evaporated and the product is resuspended in CHCl₃/MEOH/H₂O (1/2/0.8). Excess L-serine is removed by centrifugation. The product is recovered from the organic phase after the addition of 1 part CHCl₃ and 1 part water. The organic phase is taken to dryness, dissolved in CHCl₃, and applied to a 2×30 cm column of activated prewashed silica gel. The column is washed with 100 ml of CHCl₃, followed by 100 ml aliquots of CHCl₃ containing increasing MEOH. The purified product is stored in CHCl₃. Identity is confirmed by electrospray mass spectra analysis.

Coupling of SPDP-PS to Protein Carriers

SPDP-PS is coupled to purified protein after introducing additional sulfhydryls into the protein with 2-iminothiolane. Briefly, the protein is solubilized at 10 mg/ml in 10 mM Tris buffer pH 8.0 which contains 0.1 mM EDTA. A 100-fold mole excess of 2-iminothiolane is added and the reaction is allowed to proceed for 1 h (Jue et al., Biochemistry (1978) 17:5399-5405). The solution is then dialyzed overnight. To ensure the availability of maximum free sulfhydryls for coupling, the protein is reduced with 5 mM dithiothreitol (DTT). DTT is removed immediately before coupling by exclusion chromatography on a Biogel P6 column. Peak fractions are collected, and available sulfhydryls are estimated with Ellman's reagent (DTNB) (Riddles et al., Meth Enzymol (1993) 91:49-60). The reduced protein is then immediately mixed with 1 mol equivalent of SPDP-PS in 1/10 volume of ETOH. The efficiency of derivatization is estimated by measuring the release of 2-thiopyridine at 343 nm (Grassetti and Murray, Arch Biochemistry Biophys (1967) 119:41-39).

### Example 2

### Induction of Phagocytes for Prevention of Cancer

In Vivo Tumor Destruction by Bi-Functional Phosphatidylserine/Antibody Conjugate

CEA monoclonal antibody (MAb) is available from Abeam, Ltd. (Cambridge, UK). The phosphatidylserine/CEA antibody (PSCA) is synthesized as described in Example 1.

Colon tumor MC-26 cells are implanted into the splenic subcapsule of BALB/c mice. When the tumors reach a volume of 0.4-0.6 cm³, the mice are injected intravenously with either 20 μg of PSCA, 16 μg CEA MAb, 4 μg PS, a mixture of 16 μg of CEA MAb antibody and 4 μg of PS or saline. In some studies, the treatment is given 3 times, on days 0, 4 and 8. A minimum of 8 animals are treated in each group.

Animals are monitored daily for tumor measurements and body weight. Mice are sacrificed when tumors have reached a diameter of 2 cm³, or earlier if tumors show signs of necrosis or ulceration. Tumor volume is calculated according to the formula: π/6xDxd², where D is the larger tumor diameter and d is the smaller diameter. Differences in tumor growth rates are tested for statistical significance using a non-parametric test (Mann-Whitney rank sum test) that makes no assumptions about tumor size being normally distributed (Gibbons, in Nonparametric Methods for Quantitative Analysis (Gibbons, ed.), 1976, p. 160, Holt, Rinehart and Winston, New York, N.Y.).

The literature citations cited above are incorporated in pertinent part by reference herein for the reasons cited in the above text:

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of targeting a cell for phagocytosis by presenting phosphatidylserine on the surface of the cell comprising contacting the cell with a bi-functional phosphatidylserine/cell-surface recognition domain conjugate, wherein the conjugate selectively interacts with the surface of the cell by binding to a cognate moiety, thereby targeting the cell for phagocytosis.

2. The method of claim 1, wherein the phosphatidylserine is a stereoisomer.

3. The method of claim 1, wherein the cell-surface recognition domain is a polypeptide.

4. The method of claim 3, wherein the polypeptide is selected from the group consisting of antibodies, receptors, ligands and fragments thereof.

5. The method of claim 4, wherein the polypeptide is an antibody.

6. The method of claim 5, wherein the cognate moiety is an antigen.

7. The method of claim 6, wherein the antigen is selected from the group consisting of carcinoembryonic antigen (CEA), E-cadherin mutational hotspot region (ECMHR), CD17-1A antigen, CD52, CD20, HER-2/neu (c-erbB-2), CD33 and chimeric L6 antigen.

8. The method of claim 1, wherein the cell-surface recognition domain is selected from the group consisting of epidermal growth factor (EGF), transforming growth factor (TGF), urokinase plasminogen activator (uPA), transferrin, folate, adenovirus fiber, malaria cs protein, human papilloma virus capsid, lectins and fibroblast growth factor (FGF).

9. The method of claim 1, wherein the cell is a tumor cell.

10. The method of claim 1, wherein the cell is an infected cell.

11. The method of claim 1, wherein the cell-surface recognition domain is a tumor antigen.

12. A method of eliminating a cell or cells in a subject, comprising administering to the subject, a pharmaceutical composition comprising a bi-functional phosphatidylserine/cell-surface recognition domain conjugate to the subject, wherein the administering induces phagocytosis of specifically targeted cells by binding a cognate moiety, thereby eliminating the cell or cells in the subject.

13. The method of claim 12, wherein the specifically targeted cells are neoplastic cells.

14. The method of claim 12, wherein the specifically targeted cells are infected cells.

15. The method of claim 12, wherein the specifically targeted cells are immune cells.

16. The method of claim 13, wherein the neoplastic cells comprise a tumor.

17. The method of claim 14, wherein the cells are infected with a virus or a bacteria.

18. The method of claim 12, wherein the cell-surface recognition domain is a polypeptide.

19. The method of claim 18, wherein the polypeptide is an antibody.

20. The method of claim 12, wherein the cognate moiety is an antigen.

21. The method of claim 20, wherein the antigen is selected from the group consisting of carcinoembryonic antigen (CEA), E-cadherin mutational hotspot region (ECMHR), CD17-1A antigen, CD52, CD20, HER-2/neu (c-erbB-2), CD33 and chimeric L6 antigen.

22. The method of claim 13, wherein the neoplastic cells are associated with cancers selected from the group consisting of colorectal, gastrointestinal, pancreatic, prostate, lung, hematopoietic, head and neck, breast and stomach.

23. The method of claim 22, wherein the cancer is a primary tumor or metastatic cancer.

24. A pharmaceutical composition comprising a pharmaceutically acceptable carrier and a cell targeting bi-functional phosphatidylserine/cell-surface recognition domain conjugate.
